(19) United States
(12) Patent Application Publication
(10) Pub. No.: US 2012/0208192 A1
(43) Pub. Date: Aug. 16, 2012

(54) NUCLEIC ACID AMPLIFICATION EMPLOYING TEMPERATURE OSCILLATION

(75) Inventors: Martin Lee, Wiltshire (GB); Andrea Hamilton, Wiltshire (GB)

(73) Assignee: ENIGMA DIAGNOSTICS LIMITED, Wiltshire (GB)

(21) Appl. No.: 13/394,098

(22) PCT Filed: Sep. 8, 2010
(86) PCT No.: PCT/GB10/51496

§ 371 Date: Apr. 27, 2012

(30) Foreign Application Priority Data
Sep. 8, 2009 (GB) 0915664.7

(51) Int. Cl. C12Q 1/68; G01N 21/64; C12M 1/40; G01N 21/76

(52) U.S. Cl. 435/6.11; 435/287.2

(57) ABSTRACT

A method for carrying out an isothermal nucleic acid amplification reaction at a predetermined temperature, said method comprising changing the temperature of the reaction mixture away from the said predetermined temperature and allowing it to return to the predetermined temperature at least once during the amplification reaction so as to cause a temperature oscillation in particular of up to 15° C., for example of from 2-10° C. Apparatus adapted to carry out the method forms a further aspect of the invention.

The present invention relates to methods for carrying out amplification of nucleic acids as well as to apparatus for use in such methods.

Amplification of nucleic acids is an increasingly important tool in research and diagnostic applications. The methods increase the amount of specific nucleic acids present in a sample to detectable levels.

Particular examples of such methods involve thermal cycling reactions such as the polymerase chain reaction (PCR). In these amplifications, a reaction mixture is cycled between a range of predetermined temperatures to allow different phases of the reaction to be carried out cyclically. Typical predetermined temperatures include a denaturation stage (usually at about 94° C.), a primer annealing stage (usually at about 55° C.) and an extension phase (usually at about 74° C.). The reaction mixture is held at the particular temperature for just long enough to allow the phase to be carried out before the temperature is changed to effect the subsequent phase in the reaction. Such reactions are therefore complex to execute. They require complex apparatus that can effect significant temperature changes quickly and accurately. Thermal cyclers are generally programmed so as to cause the reaction mixture to “land” on the predetermined temperature required for each individual stage and hold it there for a predetermined “dwell” period before changing the temperature to that of the subsequent stage in the cycling process, and repeating the procedure until the thermal cycling procedure is complete.

Furthermore, the time taken for each heating or cooling activity adds time to the reaction, which may be disadvantageous, in particular where rapid results are required. Rapid results are frequently required, for example in circumstances such as those where health issues are at stake as in some environmental or military applications or in diagnostic applications which are conducted on site in a clinic, while a patient waits. Thermal cycling reactions can be subject to significant delays.

However, there are a large number of amplification reactions that do not require thermal cycling as such. These are collectively known as isothermal amplification methods. They include methods such as nucleic acid sequence based amplification (NASBA), strand displacement amplification (SDA), transcription mediated amplification (TMA), Loop-Mediated Isothermal Amplification (LAMP), Q-beta replicase, Rolling circle amplification, 3SR, ramification amplification (as described by Zhang et al., Molecular Diagnosis (2001) 6 No 2, p141-150) and others.

Isothermal amplification methods such as TMA have the technical advantage over PCR because they do not require thermal cycling. They therefore may require less complex apparatus. Furthermore, the process may not be subject to delays caused by the need to change the temperatures, upward and downward, to achieve the required temperature for the particular stage in the cycle.

The reactions are generally held at a single thermal set-point to effect the process, usually but not always after an initial denaturation of the template, for example to ‘melt’ or destabilise duplex DNA or to break down secondary structures in RNA. However, it has been found that such reactions may still encounter unfavourable reaction kinetics and so take a long time to complete. This can be a particular disadvantage where rapid results are required, as outlined above.

The problem is particularly acute in small reaction vessels such as capillary vessels. However, such vessels may be particularly suitable for use in these reactions both as a result of their size (they can contain the small volumes on which such reactions are usually carried out), and their optical properties as outlined further below.

Reaction vessels used in amplification reactions are generally required to have not only good thermal control properties. They should be heated in such a manner that any thermal gradients are minimised. For example, tubes that have low thermal gradients also allow precise sample temperature set points to be achieved at a resolution of less than 1° C. This facilitates powerful discriminatory assays, for example where a single base pair change in an oligonucleotide primer may drive the specificity of an amplification reaction for analysis of a single nucleotide polymorphism. Such low gradients within tubes are usually achieved through either small volumes and/or high aspect ratio vessels.

Reaction vessels must also have optical properties that make them suitable for optical acquisitions since most of these reactions are arranged so that they can be monitored as they progress, for example by means of one of the many visible signalling systems in particular fluorescent signalling systems, that are introduced into the reaction mixture.

Capillaries are one example of such a vessel. Glass capillaries and the like make ideal optical cuvettes as they may facilitate total internal reflection. A capillary cuvette provides an efficient arrangement for excitation of the sample and collection of the emission by, for example, epi-illumination.

However such vessels may not be ideal for isothermal reactions as mentioned above. Indeed the applicants have found that TMA exhibits slow reaction kinetics when carried out in a capillary vessel that is heated by means of an integral electrically conducting polymer resistance heater and which provides temperature control with high precision and accuracy.

According to the present invention there is provide a method for carrying out an isothermal nucleic acid amplification reaction at a predetermined temperature, said method comprising changing the temperature of the reaction mixture away from the said predetermined temperature and allowing it to return to the predetermined temperature at least once during the amplification reaction.

The applicants have found that by changing the temperature so as to induce a temperature oscillation or “wobble” intermittently during the amplification reaction, the overall time to completion and signal-to-noise of the assay may be improved. The applicants have surprising found that the reaction kinetics and in particular the speed of reaction is enhanced by such a procedure, even though the reaction is not at the optimum temperature throughout.

The temperature may be changed by a relatively small amount of for example no more than 15° C. and in particular from 2-10° C., such as about 5° C. in an upward and/or downward direction. In a particular embodiment, the temperature is allowed to move in a downward direction. This may promote binding or annealing of reaction components. In another embodiment, the temperature is raised slightly from the optimum during the temperature oscillation. The latter embodiment may facilitate denaturing of primer and nascent strands from the template/product, thus leading to greater stringency and/or the promotion of strand invasion processes.

In yet a further embodiment, a combination of upward and downward oscillations may be employed. In particular an upward change in temperature from the predetermined temperature is induced, and the temperature is then allowed to drop below the predetermined temperature, for instance, by a similar amount, before the predetermined temperature is restored.

In a particular embodiment, the temperature oscillation is effected more than once throughout the reaction, in particular at regular intervals. For instance, oscillations may be carried out once every few seconds or minutes depending upon the nature of the reaction being carried out. The imposed oscillations may also need only to be employed in the initial stage, or in later stages of a reaction to promote the required improvements in reaction performance. In this context, a “stage” may refer to a period of less than 50% of the total reaction time, for example less than the first 25%, or even less than the first 10% of the reaction time. Thus for example, in a forty minute reaction, oscillations of the temperature may take place throughout the 40 minute period, but even if they are effected over say the first 20 minutes or the first 10 minutes, the reaction kinetics may be enhanced.

The or each oscillation is suitably effected over a short time period, and in particular over a minimal period in that the temperature is changed by the required amount and as soon as the target temperature is reached, the system is heated or cooled to restore the reaction mixture to the set-point. How quickly this may be achieved will depend upon factors such as the nature of the apparatus including the thermal properties and capacities, the volume of the sample etc. Typically, using modern heating and cooling techniques and apparatus, the necessary temperature oscillations can be achieved in for example less than two minutes, such as less than one minute, and suitably in less than 30 seconds, for example over 5-10 seconds.

Thus for instance, the ratio of the time a reaction mixture is held at the isothermal temperature as compared to the time at which it is away from the temperature in an oscillation over the course of a reaction may be in the range of from about 20:1 to 1.5:1 for example from 10:1 to 4:1 and preferably at about 8:1.

In a particular embodiment, the oscillations are carried out at regular intervals throughout the amplification reaction. In such a regime, the reaction mixture may be held at the predetermined reaction temperature for a period of from 30 seconds to 15 minutes, for example from 4 seconds to 2 minutes, and then an oscillation effected.

Furthermore, the method provides improvements in reaction kinetics even where the isothermal amplification reaction is carried out in optically favourable vessels such as capillary type cuvettes as outlined above.

Without being bound by theory, it is possible that by inducing temperature oscillation around the set-point, there may be advantages to ensuring the presence of an in-tube gradient that facilitates eddies (mixing) within the reaction mixture, and so ensures that the necessary nucleic acid interactions take place. If the in-tube gradient is too low, then there may be insufficient destabilisation and re-association of nucleic acids within a reaction vessel to allow the amplification reaction to progress efficiently. The reaction may exhibit slow kinetics.

For example, a standard micro-amp tube with a 40 μl volume at thermal equilibrium is expected to have a 7° C. gradient, whilst a capillary vessel may have a gradient of as little as 0.1° C. However, as illustrated below, the applicants have found that the method results in improvements in reaction kinetics when reactions are carried out in both capillary vessels containing small reaction volumes (25 82 l). Larger cuvettes containing larger reaction volumes (100 μl) may also exhibit improvements.

By ensuring that isothermal amplification reactions are efficiently carried out in such vessels, the method described herein means that the reaction vessel may be selected principally on the basis of its optical properties. Thus a particularly suitable reaction vessel for carrying out the method of the invention is a capillary vessel, in particular a glass capillary vessel. Other capillary vessels may be constructed from metal or plastics materials as well as glass or combinations thereof.

This is particularly useful where the reaction is carried out using a visible signalling system such as a fluorescent signalling system, as it means that the method can be used during many of the available “real-time” monitoring applications. In general, in such methods, a visible signal is generated as an amplification reaction progresses. In particular, a fluorescent reagent is present in the amplification reaction mixture and the system is set up so that, when the vessel is illuminated, a fluorescent signal is generated or modified as the amplification reaction progresses. By monitoring the fluorescent signal during the amplification reaction, the course of the amplification may be monitored and the information obtained may be used for example to quantitate the amount of nucleic acid present in the sample at the beginning of the reaction.

Suitably, where the amplification is carried out in the presence of a fluorescent reagent as described above, it is preferable that optical measurements are taken after the temperature change or oscillation has taken place and the reaction mixture returned to the set-point for a sufficient period of time to allow an equilibrium to be reached. This may be preferable in particular where fluorophores such as fluorescein are used, since these can exhibit changes in fluorescence as a result of thermally induced pH changes with the medium, which contains electrolytes.

Suitable isothermal amplification reactions that may be enhanced by use of the method of the invention include nucleic acid sequence based amplification (NASBA), strand displacement amplification (SDA), transcription mediated amplification (TMA) which is specific for RNA targets, Loop-Mediated Isothermal Amplification (LAMP), Q-beta replicase, Rolling circle amplification, 3SR, ramification amplification and others.

Suitable visible signalling systems include those known in the art. The precise selection of visible signalling reagents such as fluorescent reagents used in any particular isothermal reaction will depend upon the nature of the reaction being effected, the nature of the sample etc. However, signalling systems may utilise agents in particular dyes, that intercalate or otherwise interact with nucleic acids and generate a differential signal such as a fluorescent signal, depending upon whether or not that are interacting with a nucleic acid. Examples of such dyes include SYBR Green™ such as SYBR Green I, SYBR Gold, ethidium bromide, YOPRO-1, and the SYTO dyes including green dyes such as SYTO 9 and red SYTO dyes such as SYTO® 17, SYTO® 59, SYTO® 60, SYTO® 61, SYTO® 62, SYTO® 63 and SYTO® 64.

Certain visible signalling systems utilise oligonucleotide probes or primers that bind specific target nucleic acids, and are labelled with a visible label such as a fluorescent label. Examples of suitable fluorescent labels include for example fluorescein or fluorescein derivatives such as FAM (5 or 6-carboxyfluorescein), TET (6-carboxy-4′,5′-dichloro-2′,4,7,7′-tetrachlorofluorescein), JOE (6-carboxy-4,5-dichloro-2,7-dimethoxyfluorescein, HEX (succinimidyl ester of 6-carboxy-2′,4,4′,5′,7,7′-hexachlorofluorescein) or fluorescein isothiocyanate, Alexafluor™ dyes such as Alexafluor-594, rhodamine or derivatives such as rhodamine B, rhodamine 123, TAMRA (N,N,N′,N′-tetramethyl-6-carboxyrhodamine, ROX (6-carboxy-X-rhodamine), R6G (6-carboxyrhodamine), sulphorhodamine B, sulphorhodamine 101, lissamine rhodamine B sulphonyl chloride, tetramethylrhodamine isocyanate as well as other fluorophores such as ethidium, coumarin and derivatives such as Coumarin 120 (7-amino-4-methyl-coumarin), Coumarin 151 (7-amino-4-trifluoromethylcoumarin), acridine and derivatives such as acridine isothiocyanate, Lucifer yellow (4-amino-N-3-vinylsulphonyl)phenyl napthalimide-3,5-disulphonate, N-(4-anilino-1-naphthyl)maleimide, anthranilamide, 5′.5″-dibromopyrogallol-sulphonaphthalein (Bromopyrogallol red), eosin, esosin isothiocyanate, and others.

Particularly useful labels in this context are fluorescein or fluorescein derivatives such as carboxyfluorescein compounds , such as 5-carboxyfluorescein, 6-carboxyfluorescein, or their succinimidyl esters, cyanine dyes or rhodamine dyes. Particular examples of such dyes include fluorescein, JOE, FAM, HEX, TET, TAMRA, ROX Cy5, Cy3, Cy5.5,BoDIPY FL, rhodamine, rhodamine green, rhodamine red, Oregon Green 488, 500 or 514, Texas red, LightCycler Red 610, 640, 670 or 705.

Signalling systems of this type may also involve the use of so called “quencher” moieties. These interact with fluorophores and absorb fluorescent energy from them, so they act as acceptors of fluorescent energy. If the system is arranged so that the physical separation of the fluorophore and the quencher differs depending upon the presence of amplification product, the change in fluorescence can be used to characterise the amplification. Quencher molecules may themselves be fluorescent, provided they act as fluorescent energy ‘acceptors’ or they may be “dark quenchers” that do not generally fluoresce, or do not do so in the conditions encountered during the particular reaction. This will generally involve illumination of the system with light of a wavelength sufficient to ensure that the fluorescent label is excited and emits fluorescence at a characteristic wavelength that can then be monitored.

Where a signalling system relies on fluorescence energy transfer between donor and acceptor or quencher moieties, these are often referred to as FET or FRET systems.

There are two commonly used types of FET or FRET probes, those using hydrolysis of nucleic acid probes to separate donor from acceptor, and those using hybridisation to alter the spatial relationship of donor and acceptor molecules.

One particular assay for monitoring the progress of an amplification reaction is the Taqman™ assay as described in U.S. Pat. No. 5,538,848. This assay utilises hydrolysis probes consisting of DNA oligonucleotides, which are labelled with donor and acceptor molecules. The probes are designed to bind to a specific region on one strand of a target sequence or amplification product. Following annealing of an amplification primer to this strand, a polymerase enzyme such as Taq enzyme extends the DNA with 5′ to 3′ polymerase activity. Taq enzyme also exhibits 5′ to 3′ exonuclease activity. TaqMan™ probes are protected at the 3′ end by phosphorylation to prevent them from priming Taq extension. If the TaqMan™ probe is hybridised to the product strand than an extending Taq molecule may also hydrolyse the probe, liberating the donor from acceptor as the basis of detection. The signal in this instance is cumulative, the concentration of free donor and acceptor molecules increasing with each cycle of the amplification reaction.

Hybridisation probes are available in a number of guises. Molecular beacons or molecular torches are oligonucleotides that have complementary 5′ and 3′ sequences such that they form hairpin loops. Terminal labels, one of which is a fluorescence donor and one of which is a quencher are provided on the beacon structure and so are in close proximity for FRET to occur when the hairpin structure is formed. Following hybridisation of molecular beacon to a complementary sequence the fluorescent labels are separated, so FRET does not occur, and this forms the basis of detection. Similarly, in the case of molecular torches (U.S. Pat. No. 6,534,274), the hairpin structure is made up of a target binding domain and a target closing domain, held together in the form of a hairpin by a joining region. The target binding domain is biased towards the target sequence and forms a more stable hybrid with that sequence than with the target closing domain. Thus, when in the presence of the target, the hairpin is opened so that a target binding domain of the oligonucleotide only hydridises to the target. This means that another portion (a target closing domain) of the torch that does not hybridise to the target and remains single stranded. However, it is clearly now separated from the other member of the donor/quencher pair, and so the levels of fluorescence are different generating a detectable signal. Such systems are used in isothermal amplification detection techniques, in particular TMA.

Pairs of labelled oligonucleotides may also be used. These hybridise in close proximity on an amplification product strand, bringing fluorescent donor and quencher molecules together so that FRET can occur.

A signalling system which utilises a combination of a single labelled probe and an intercalating dye, is described in WO 99/28500.

WO99/42611 describes an assay for detecting the presence of particular nucleic acid sequences that may be adapted to quantify the amount of the target sequence in the sample. In this assay, an amplification reaction is effected using a set of nucleotides, at least one of which is fluorescently labelled. Thus the amplification product has fluorescent label incorporated in it. The reaction is effected in the presence of a probe, which can hybridise to the amplification product and which includes a reactive molecule able to absorb fluorescence from or donate fluorescent energy to said fluorescently labelled nucleotide. The reaction can then be monitored by measuring the fluorescence of said sample as this will alter during the course of the reaction as more amplicon product is formed which hybridises to the probe and gives rise to a FET or FRET interaction between them.

Using these techniques, it is possible to directly detect not only whether a particular target sequence is present within a sample (because amplification is taking place), but by appropriate manipulation of the data, to quantify the amount or determine when an amplification reaches completion. In particular, graphs plotting fluorescence against time or cycle number generally produces a sigmoidal plot peak, which rises steeply as amplification progresses and the amount of DNA in the sample increases, but which then reaches a plateau as amplification becomes saturated.

It has also been found that the properties of labels may be different depending upon their position within a nucleic acid sequence, and in particular whether they are double or single stranded and located towards a terminus of an oligonucleotide, or positioned internally. The changes in the properties of the label when say it is attached to an end region of an oligonucleotide or primer, as compared to when it has been incorporated into a DNA sequence can itself give rise to a detectable signal. The principles and examples of how the technique operates are illustrated for example in WO/0079009 and WO02/057479.

The LUX (Light Upon eXtension) PCR chemistry is a particular example of this type of assay, and is known for example from Nazarenko et al., Nucleic Acids Res. 2002, 30(9) e 37, and Nucleic Acids Res. 2002, 30(9) p2089-2195, and exemplified for instance by Aitcihou et al. Molecular and Cellular Probes 19 (2005) 323-328. The technique is based upon the use of an oligonucleotide as a primer, which includes a modification so that it is detectable when hybridised to a complementary strand as compared to when it is free. In the usual mode, the method uses an oligonucleotide primer labelled with a single fluorescent moiety, to monitor nucleic acid amplification in the absence of traditional DNA-intercalating dye (such as SYBR dyes) or fluorescent probes. The primer used in LUX chemistry generally resembles that of a molecular beacon probe in that it relies on the formation of a hairpin motif to facilitate intra-molecular quenching in the absence of a target molecule. For example a LUX primer has a 5′ motif (which is usually about 5 nucleotides in length) complementary to its 3′ target specific priming terminus. When folded, the fluorescent moiety, which is generally bound to or close to a residue at the 3′ end of the primer, is quenched via base-pairing or proximity to a residue, in particular a guanosine (dG) at the 5′ end, and to some extent also by the nearest neighbour in the stem region. Generally, the label is attached to a cytosine (C) residue or a thymine (T) residue.

The reason for this is believed to be associated with the occurrence of a process of photoinduced electron transfer (PET). In the LUX process, a 3′ amino-dC label is brought into close proximity with a 5′ guanosine residue. The conformational microenvironment formed allows guanosine to behave as a ground state electron donor. This means that the near-by fluorophore fails to achieve the high-energy electron state required for photon emission. As such, quantum yield is decreased, thus quenching the dye.

When the guanosine is located internally of a nucleotide sequence, for example following extension of the primer and generation of the amplicon, the microenvironment of the fluorophore changes. The electron-donor effect is dissipated along the sequence, making the neighbouring guanosine residues poorer electron donors, and so “de-quenching” the fluorophore by up to six fold. This change is sufficient to allow the real-time monitoring of the PCR reaction.

Preferably the terminal sequence carrying the label, for example of the hairpin has a “blunt end” as the presence of an overhang has been found to quench the fluorescence less efficiently.

In the presence of target nucleic acid in the sample, the primer binds to a target recognition sequence (in the case of a hairpin, after linearisation), and becomes extended so the label becomes internal within the sequence. As a result it has been found that the quenching is reduced. The primer also acts as a conventional PCR primer and so the fluorescent moiety becomes incorporated into the amplicon. As the fluorescent moiety is not quenched, at least not to the same extent in the amplicon as compared to the hairpin primer, the existence of amplicon may be detected directly.

Examples of suitable quencher moieties include Methyl red, which is commonly used for example in “Scorpion” probes. Other examples include those described in U.S. Pat. No. 6,323,337, the content of which is incorporated herein by reference. Typically, the quenching moiety is a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a coumarin (including hydroxycoumarins and aminocoumarins and fluorinated and sulphonated derivatives thereof (for example as described in U.S. Pat. No. 5,830,912 and U.S. Pat. No. 5,696,157, the content of which are incorporated herein by reference), a polyazaindacene (for example as described in U.S. Pat. No. 4,774,339, U.S. Pat. No. 5,187,288, U.S. Pat. No. 5,248,782, U.S. Pat. No. 5,274,113, 5,433,896, U.S. Pat. No. 6,005,113, the content of all of which are incorporated by reference), a xanthene, an oxazine or a benzoxazine, a carbazine (U.S. Pat. No. 4,810,636, the content of which is incorporated by reference), or a phenalenone or benzphenalenone (such as those described in U.S. Pat. No. 4,812,409, the content of which is incorporated herein by reference).

Other quenching moieties that are essentially nonfluorescent dyes include in particular azo dyes (such as DABCYL or DABSYL dyes and their structural analogues), triarylmethane dyes such as malachite green or phenol red, 4′,5-diether substituted fluoresceins (as described for example in U.S. Pat. No. 4,318,846, or asymmetric cyanine dye quenchers (as described for example in WO 99/37717, the content of each of which is incorporated herein by reference.

A particular quencher moiety is DABCYL (4-(dimethylaminoazo) benzene-4-carboxylic acid) or a derivative thereof, such as the halide or amide derivative, which facilitates attachment of the moiety to an amino acid of an oligonucleotide.

Other examples of quenchers are non-fluorescent derivatives of 3- and/or 6-amino xanthene that is substituted at one or more amino nitrogen atoms by an aromatic or heteroaromatic ring system (for example as described in U.S. Pat. No. 6,399,392, the content of which is incorporated herein by reference). These quenching dyes typically have absorption a maximum above 530 nm, have little or no observable fluorescence and efficiently quench a broad spectrum of luminescent emission, such as is emitted by chemilumiphores, phosphors, or fluorophores. In one embodiment, the quenching dye is a substituted rhodamine. In another embodiment, the quenching compound is a substituted rhodol.

For example, where the isothermal reaction is a TMA reaction, it will generally be effected in the presence of a probe in the form of a molecular torch, that at least partially hybridises to the RNA amplification product. The torch is provided with a quencher moiety at one end and a fluorescent label at the other. In the absence of amplification product, the complementary regions of the beacon hybridise together and so the quencher and the fluorescent label are in close proximity to each other. As a result, fluorescence from the fluorescent label is quenched. However, in the presence of the amplification product, the torch will hybridise to the product to form an “open” structure, in which the quencher and the fluorescent label are separated. As a result, the fluorescent signal is no longer quenched and the increase in fluorescence can be detected.

An alternative signalling system may rely on bioluminescence as the basis of signalling. For example, WO 02/090586 describes a system that utilises an RNA probe, to form the basis of a bioluminescent system. In that method, a target DNA sequence is contacted with an RNA probe under conditions such that the probe will bind to the sequence. It is then subjected to conditions under which RNA probe bound to nucleic acid in the form of an RNA/DNA duplex is hydrolysed to generate adenosine monophosphate (AMP). The AMP produced is then detected, suitably by phosphorylating it to adenosine triphosphate (ATP) enzymatically either directly or by way of the production of adenosine diphosphate, which is then detected using a bioluminescent system such as the luciferin/luciferase system. Examples of the application of such detection systems are described for example in WO 96/02665. Bioluminescent signal produced can be related to the presence or nature of the target nucleic acid sequence in the sample.

RNA probes may be readily hydrolysed by a variety of enzymes, when in double stranded form. These include polymerase enzymes commonly used in PCR reactions such as Taq polymerase. Alternatively they may be hydrolysed by RNAse enzymes, which will hydrolyse them only when in the form of RNA/DNA duplexes. This may be effected using an endonucleolytic activity such as that provided by the enzyme known as RNAse H which may be used in combination with an oligoribonuclease.

Bioluminescent systems such as the luciferase/luciferin system do react with the deoxyATP (dATP) which is usually added to amplification reactions in order to obtain the polymerase activity required, but the catalytic efficiency is lower and the light produced is red rather than the yellow-green emissions obtained from ATP. Thus, they will be able to distinguish between ATP produced as a result of hydrolysis of the RNA probe and any dATP which may be required to be added to the reaction mixture for other purposes or produced from hydrolysis of DNA rather than RNA. Bioluminescent detection systems may be more sensitive than fluorescent systems.

A further aspect of the invention comprises apparatus for carrying out a method as described above, said apparatus comprising means for controlling the temperature of a reaction vessel, wherein said means is arranged to cause at least one temperature oscillation as defined above during the method. The precise form of the apparatus may vary depending upon the particular heating technology being used.

If required, the apparatus may comprise a conventional thermal cycler such as those used in thermal cycling amplification reactions such as the polymerase chain reaction, which are modified in that they are programmed to produce the required constant temperature with the oscillations as defined above. Such devices are capable of pre-programmed thermal oscillations that do not require a set “dwell” period for any given period. In particular, in one embodiment of the invention, the apparatus is programmed so that the thermal oscillation has a smooth or even thermal waveform. In other words, there is no “dwell” period during the oscillation itself, but the temperature is varied continuously away from and back to the predetermined temperature of the isothermal reaction.

In this instance, the programming can be such that the nature of the temperature oscillation or “wobble” is defined to achieve the desired enhancement in the reaction.

Frequently such apparatus includes means for monitoring the reaction as they progress to allow reaction kinetics to be obtained and used, for instance to allow quantification of the target to be undertaken. Thus they may incorporate detectors such as fluorimeters.

The apparatus of the invention may, in a particular embodiment, contain a feedback loop that controls and creates oscillations as a thermal waveform, which matches the particular hybridisation kinetics of the reaction being effected. This could be determined by monitoring the progress of the reaction as outlined above and feeding that information back to the temperature control system, so that any reduction in the rate of isothermal reaction, as noted for instance by a reduction in the expected rate of amplification of target as indicated by a signal such as a fluorescent signal, leads to the generation of an oscillation.

However, the range of temperatures that is required to be achieved is generally much smaller than where significantly different temperatures are required to effect different stages in the reaction. The small deviations that are required to effect the method described above may for instance be achieved using conventional thermostats that are programmed to allow the required level of variation before restoration of the optimum reaction temperature is achieved. Many fluorimeters may conventionally be provided with heating means, such as the plate fluorimeters developed for immunoassay purposes, and these also may be suitable for carrying out the method described above if programmed appropriately.

For instance, fluid heaters and coolers such as air heaters and coolers in particular those heated by halogen bulbs, as described for example in U.S. Pat. No. 6,787,338 and WO2007/054747, the content of which is incorporated by reference, as well as in vessels using ECP as resistive heating elements, for example as described in WO 98/24548 and WO 2005/019836 are usable in the method and so may form an element of the apparatus. The apparatus may also use more conventional devices such as solid block heaters that are heated by electrical elements. For cooling the apparatus may incorporate thermoelectric devices, compressor refrigerator technologies, forced air or cooling fluids as necessary.

Preferably, the apparatus use has the facility to induce rapid changes in temperature in the reaction vessel. This ensures that the temperature oscillations can be effected quickly, so that the benefits are achieved, with minimum loss of optimum reaction time. Therefore, turbulent air arrangements such as the Corbett RotorGene® may be utilized. In a particular embodiment, the heating technology is based upon an electrically conducting polymer that acts as a controllable resistance heater. Suitably the reaction vessel is contiguous with or comprises the electrically conducting polymer (ECP). Examples of reactions vessels of this type are described in WO 98/24548 and WO 2005/019836.

The reaction will now be particularly described by way of example with reference to the accompanying drawings in which:

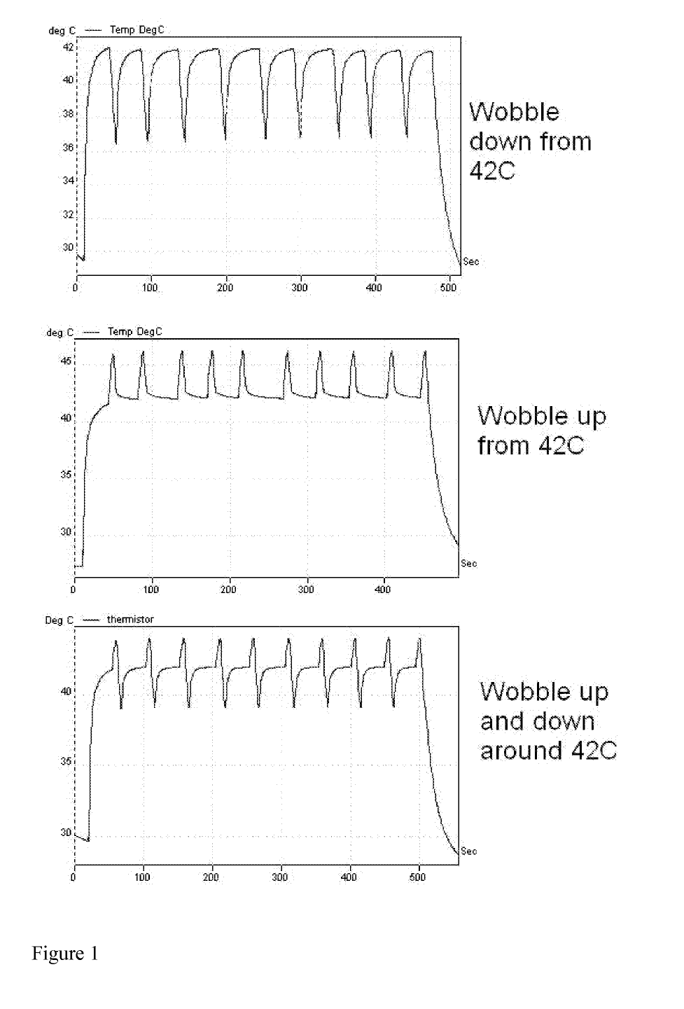

FIG. 1 shows PicoLog traces of thermal oscillations that may be applied during a TMA reaction. The measurements are from a miniature thermistor (supplied by GE) placed in an ECP capillary tube in a mock reaction with the reaction mix replaced by the same volume of water. The first, upper trace, labelled “Wobble down from 42C”, shows the reaction temperatures that were obtained with the temperature regime used in the following two figures, whilst the middle and lower traces respectively show upwards and upwards+downwards excursions that may also be employed;

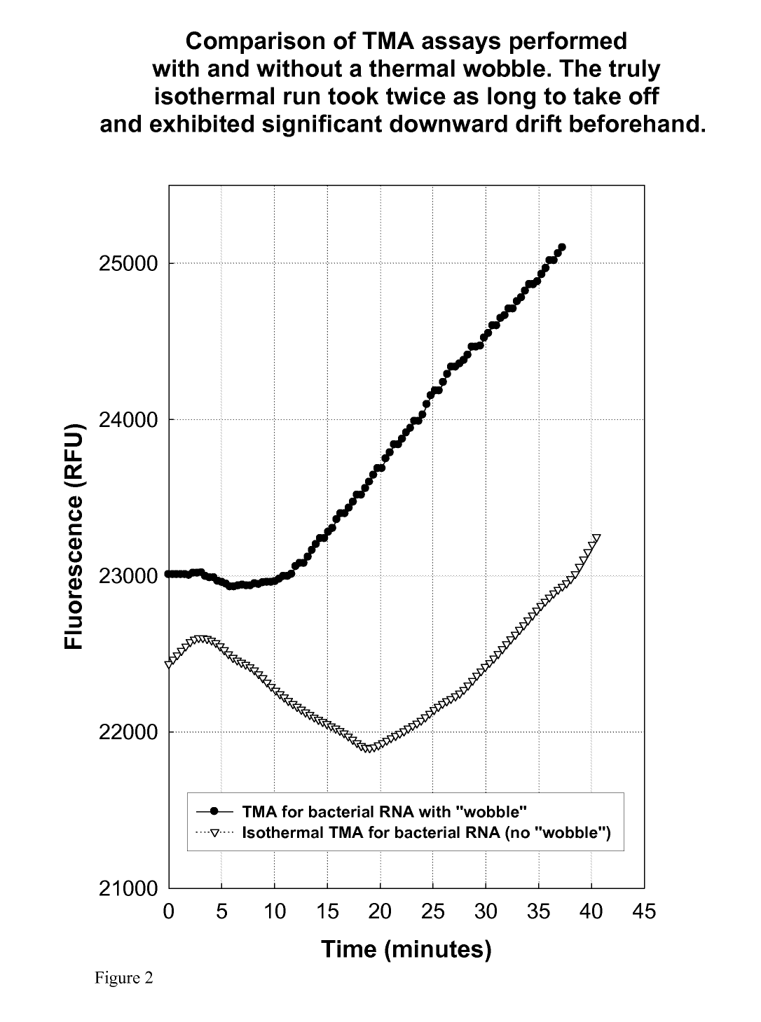

FIG. 2 shows a comparison of the results of a conventional TMA of bacterial RNA and a method of the invention; and

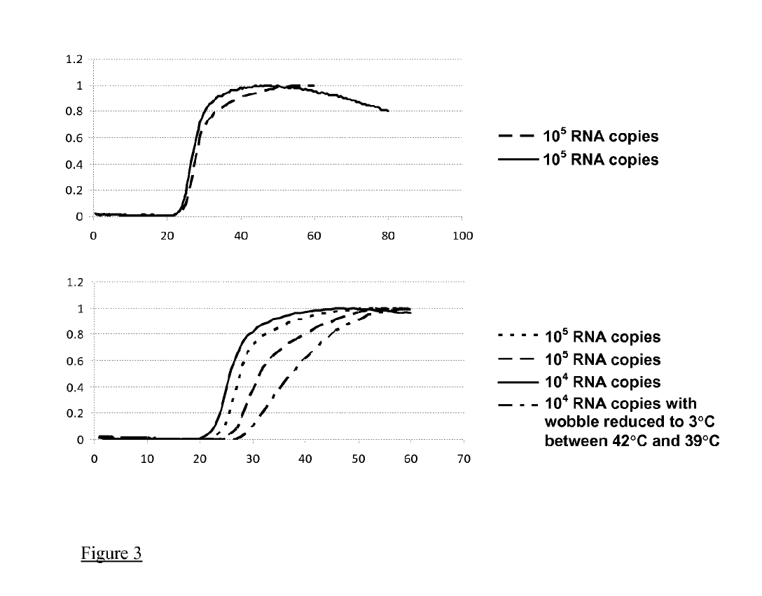

FIG. 3 shows the results of a series of TMA reactions carried out using the method of the invention.

### EXAMPLE 1

### Amplification and Detection of Bacterial RNA Using Isothermal TMA

A number of conventional TMA reactions for bacterial RNA were carried out using an Enigma FL apparatus (Enigma Diagnostics Limited, UK). In this case, the reaction vessel is a capillary tube of 25 μl capacity, heated by electrically conducting polymer. The apparatus is capable of carrying out PCR but on this occasion, it was programmed to carry out an isothermal amplification at 42° C. for 40 minutes. In some cases, a thermal oscillation or wobble was effected throughout the reaction as illustrated in FIG. 1 (upper trace). The reactions were monitored continuously by measuring fluorescence from a molecular torch specific for the bacterial DNA being amplified. .

The results are shown in FIG. 2 where the results of a TMA performed at a constant 42° C. for 40 minutes (lower trace) are compared with the results (upper trace) of a TMA subjected to a “wobbled” temperature profile of 42° C. for 20 seconds followed by 37° C. for 5 seconds for a total of 80 cycles, and so also taking about 40 minutes. The results show that in the case of upper trace (where the temperature was wobbled), the “take off” point for the amplification (arrowed) began sooner, after about 12 minutes (equivalent to 25 oscillations), as compared to about 18 minutes (also arrowed) for the truly isothermal, constant temperature version in the lower trace. Thus effectively, the wobble resulted in the reaction “taking off” about twice as fast. However, additionally, the truly isothermal reaction in the lower trace showed significant downwards drift before the amplification became apparent.

### EXAMPLE 2

### Confirmatory Experiments.

The method of Example 1 was repeated a number of times on the Enigma FL instrument using the method of the invention. The target bacterial RNA was added at a level of either 10⁵ copies or 10⁴ copies. The induced thermal oscillations (wobbles) were 5° C. in amplitude (from 42° C. to 37° C.) except for one, where it was reduced to 3° C. (from 42° C. to 39° C.). The results are shown in FIG. 3.

Given that this is an “isothermal” reaction supposed to be performed at 42° C., the lack of deleterious effect of compressing the temperature regime away from what is expected to be the optimum is surprising.

1. A method for carrying out an isothermal nucleic acid amplification reaction at a predetermined temperature, said method comprising changing the temperature of the reaction mixture by up to 15° C. from the said predetermined temperature and allowing it to return to the predetermined temperature at least once during the amplification reaction so as to cause a temperature oscillation.

2. The method according to claim 1 wherein the temperature is changed by from 2-10° C.

3. The method according to claim 2 wherein the temperature is change by about 5° C. in an upwards and/or a downwards direction.

4. The method according to claim 1 wherein the temperature oscillation is effected more than once throughout the reaction.

5. The method according to claim 4 wherein temperature oscillations are effected at regular intervals throughout the reaction.

6. The method according to claim 1 wherein the amplification reaction is carried out in a capillary reaction vessel.

7. The method according to claim 1 wherein the amplification reaction is effected in the presence of a visible signalling system.

8. The method according to claim 7 wherein the visible signalling system is a fluorescent signalling system.

9. The method according to claim 7 wherein the visible signalling system is a bio luminescent signalling system.

10. The method according to claim 7 wherein optical measurements are taken after the temperature oscillation has taken place and the reaction mixture returned to the predetermined temperature for a sufficient period of time to allow an equilibrium to be reached.

11. The method according to claim 1 wherein the isothermal amplification reaction is selected from nucleic acid sequence based amplification (NASBA), strand displacement amplification (SDA), transcription mediated amplification (TMA), Loop-Mediated Isothermal Amplification (LAMP), Q-beta replicase, Rolling circle amplification, 3SR or ramification amplification.

12. The method according to claim 11 wherein the isothermal amplification reaction is a TMA.

13. The method according to claim 12 wherein the reaction is effected in the presence of a probe in the form of a molecular beacon or molecular torch.

14. Apparatus for carrying out a method according to claim 1, said apparatus comprising means for controlling the temperature of a reaction vessel, wherein said means is arranged to cause at least one temperature oscillation as defined in claim 1 during the method.

15. Apparatus according to claim 14 wherein the apparatus comprises electrically conducting polymer arranged to for a resistance heater to the contents of a reaction vessel.

16. Apparatus according to claim 14 wherein the apparatus is arranged to monitor progress of an isothermal reaction being carried out, and produce a temperature oscillation in response to a reduction in the rate of reaction.

17. Apparatus according to claim 14 wherein the apparatus is programmed so that the temperature of the reaction mixture during oscillation shows an even thermal waveform without any “dwell” period at any specific temperature.
